(19) United States
(12) Patent Application Publication
(10) Pub. No.: US 2011/0144346 A1
(43) Pub. Date: Jun. 16, 2011

(54) METHOD FOR PRODUCING N-PHENYL-N-(4-PIPERIDINYL) AMIDE SALTS

(75) Inventors: Oliver Flogel, Winterthur (CH); Ulrich Weigl, Hilzingen (DE)

(73) Assignee: CILAG AG, Schaffhausen (CH)

(21) Appl. No.: 13/002,376

(22) PCT Filed: Jul. 3, 2008
(86) PCT No.: PCT/EP2008/005418

§ 371 Date: Mar. 2, 2011

(51) Int. Cl. C07D 211/66

(52) U.S. Cl. 546/224

(57) ABSTRACT

The invention relates to a method for producing N-phenyl-N-(4-piperidinyl)amide salts, particularly pharmaceutically tolerable addition salts of the compound Remifentanil, in that a compound of the formula (III) is reacted with an acrylic acid alkyl ester of the formula CH₂═CH—C(O)—OR: where independently of each other R denotes low-molecular alkyl, preferably (C₁₋₄)alkyl, preferably methyl or ethyl, R₁ denotes low-molecular alkyl, preferably (C₁₋₄)alkyl, preferably methyl or ethyl; and HX denotes an inorganic or organic acid, wherein the components are optionally reacted in the presence of a catalyst, preferably at a higher temperature, thereby obtaining the salt of the compound of formula (I).

### CROSS-REFERENCE TO RELATED APPLICATIONS

This application is the National Stage of International Application No. PCT/EP2008/005418, filed Jul. 3, 2008, the contents of which is incorporated by reference herein.

### TECHNICAL FIELD

The present invention relates to the preparation of selected N-phenyl-N-(4-piperidinyl)amide salts, in particular salts of the compound remifentanil, in particular remifentanil hydrochloride.

### PRIOR ART

EP 0 383 579, Glaxo Welcome Inc., published on 22 Aug. 1990, describes N-phenyl-N-(4-piperidinyl)amides and their salts, as well as the preparation thereof. There are described in particular compounds of formula (I) and their salts:

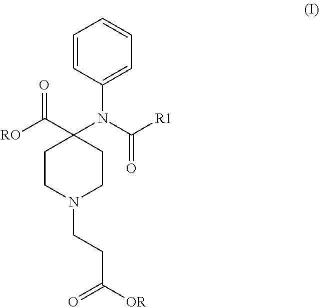

wherein R and R₁ independently of one another denote low-molecular alkyl. Remifentanil corresponds to the compound of formula (I) wherein R denotes methyl and R₁ denotes ethyl.

Compounds of formula (I) are synthetic opioids which are used as anaesthetics. A preferred method for preparing the salts of compounds of formula (I) consists in alkylating a compound of formula (II) by means of an aza-Michael addition and converting the resulting compound of formula (I) into a pharmaceutically acceptable salt, preferably into the hydrochloride:

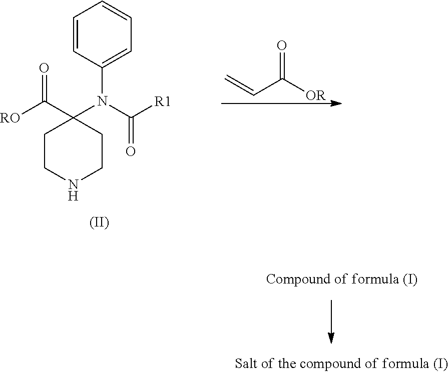

The process of aza-Michael addition, starting from the free amine of a compound of formula (II) in the presence of a Michael acceptor, such as, for example, an acrylic acid derivative [e.g. CH₂═CH—C(O)—OR], has significant disadvantages. Thus, according to J. Med. Chem. 32, page 968 (1989), an intramolecular displacement of the R₁—C(O) substituent on the secondary piperidine nitrogen atom has been observed in the reaction of the piperidine compound of formula (II), which results in undesirable secondary products that are difficult to remove.

Furthermore, the compounds of formula (I) are obtained in liquid form and are purified by chromatography and then converted into a salt, which is expensive.

### DESCRIPTION OF THE INVENTION

It has now been found that it is possible, surprisingly, first to convert the compound of formula (II) into a salt and to convert that salt [referred to hereinbelow as compound (III)] directly into the desired salt of the compound of formula (I) by means of an aza-Michael addition, the salt of the compound of formula (I) so obtained being obtained directly in pure form. This one-stage process is simple to carry out and additionally has the surprising advantage that no intramolecular displacement of the R₁—C(O) substituent on the secondary piperidine nitrogen atom takes place. In addition, no other undesirable secondary reactions occur, such as, for example, saponification of the ester groups present in the compound of formula (III) or dimerisation reactions, in which two molecules of the compound (II) dimerise by conversion of the ester group of one molecule into an amide by attack of the free piperidine nitrogen. These properties are particularly important because the compounds of formula (I) belong to the class of highly potent active ingredients, and any additional outlay in their preparation is associated, for safety reasons, with particular conditions and makes the process considerably more expensive.

It is also surprising that the process according to the invention proceeds without the addition of a catalyst, it being possible, in order to accelerate the reaction, to add a catalytic amount of a catalyst, for example of a base, to the reaction mixture. The pure compound of formula (I) can, if required, subsequently be obtained from the salt in a manner known per se.

The present invention is defined in the patent claims. In particular, the present invention relates to a process for the preparation of N-phenyl-N-(4-piperidinyl)amide salts, in particular pharmaceutically acceptable addition salts of the compound remifentanil, in particular remifentanil hydrochloride, which process is characterised in that a compound of formula (III) is reacted with an acrylic acid alkyl ester of the formula CH₂—CH—C(O)—OR:

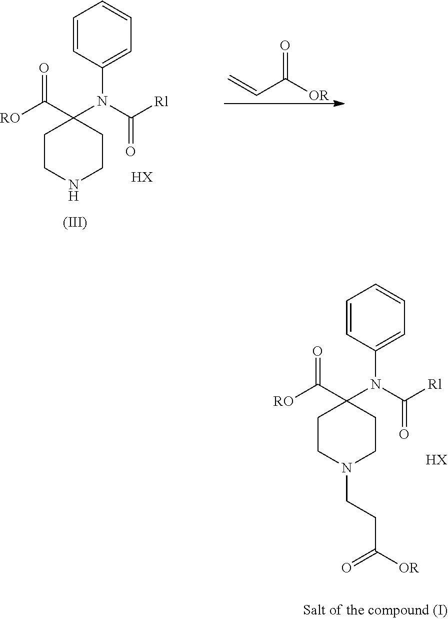

wherein
the substituents R independently of one another denote low-molecular alkyl, preferably (C₁₋₄)-alkyl, preferably methyl or ethyl, preferably methyl;
R₁ denotes low-molecular alkyl, preferably (C₁₋₄)-alkyl, preferably methyl or ethyl, preferably ethyl; and HX denotes an inorganic or organic acid, preferably hydrogen halide, preferably HBr, HI, HCl, preferably HCl; or an organic mono- or di-carboxylic acid, preferably oxalic acid;
wherein the components are optionally reacted in the presence of a catalyst, preferably at elevated temperature, and then the reaction mixture is allowed to cool, the salt of the compound of formula (I) being obtained.

The present invention relates also to the salts of the compound of formula (I) prepared according to the invention, which salts are obtained directly in crystalline or amorphous form.

The salts of the compound of formula (I) prepared according to the invention can also be used to prepare the free base of the compound of formula (I) by converting a salt of the compound of formula (I) prepared according to the invention into the free base of the compound of formula (I) in a manner known per se.

The present invention relates also to a process for the preparation of the free base of the compound of formula (I), which process is characterised in that a salt of the compound of formula (I) prepared according to the invention is converted into the compound of formula (I) in a manner known per se.

Preferably, remifentanil hydrochloride is prepared, wherein in the compound of formula (III): R=methyl, R₁=ethyl and HX═HCl.

An important advantage of the process according to the invention is that the compound of formula (III) is in the form of a crystalline or amorphous solid and yields the end product in a single stage. In this respect, the compound of formula (III) can be reacted directly with an acrylic acid alkyl ester of the formula CH₂═CH—C(O)—OR and the acrylic acid alkyl ester, preferably acrylic acid methyl ester, can be used as solvent. It is preferred, however, to use a suitable inert solvent which contains the acrylic acid ester in an amount of approximately from 1 to 10 equivalents, calculated on the amount of compound of formula (III).

There is used as a suitable inert solvent for the reaction of the compound of formula (III) with the acrylic acid alkyl ester of the formula CH₂═CH—C(O)—OR preferably an alcohol, such as methanol, ethanol, n-propanol, isopropanol, butanol; or an ether, for example tert-butyl methyl ether; or tetrahydrofuran (THF); or acetonitrile; or another compound suitable as solvent, or a mixture of those compounds. The choice of a suitable inert solvent or of a mixture of such solvents can readily be optimised by the person skilled in the art.

The compound of formula (III) is thereby dissolved or suspended in the inert solvent or in the mixture of inert solvents, and at least an equimolar amount of acrylic acid alkyl ester, calculated on the molar amount of the compound of formula (III), and optionally also a catalyst, is added thereto. The acrylic acid ester is preferably used in an amount of approximately from 1 to 10 equivalents, preferably in an amount of approximately from 3 to 10 equivalents, preferably in an amount of approximately from 3 to 6 equivalents, and in particular in an amount of approximately 5 equivalents, calculated on the amount of compound of formula (III). The weight ratio of solvent to acrylic acid alkyl ester is preferably in the range from 8:2 to 2:8 and preferably in the range from 6:4 to 4:6.

The reaction mixture is stirred for a sufficiently long time at elevated temperature, whereby the compound of formula (I) is obtained in crystalline or amorphous form immediately and/or upon cooling of the reaction mixture and can be filtered off. Preferably, the reaction mixture is treated at a temperature in range of approximately from 20 to 120° C., depending on the boiling point of the reaction mixture, preferably at a temperature in the range of approximately from 50 to 80° C., for approximately from 1 hour to 48 hours, preferably for approximately from 4 to 6 hours. The salt of the compound (I) that forms is generally obtained in crystalline or amorphous form immediately or upon cooling of the reaction mixture.

Preferably, a catalyst is added to the reaction mixture. The addition of a suitable catalyst accelerates the rate of reaction markedly. Suitable catalysts are, for example, metal carbonates, such as, for example, sodium carbonate, potassium carbonate, lithium carbonate, magnesium carbonate, calcium carbonate; tertiary amines, such as, for example, triethylamine, N-methylmorpholine, Hünig base (ethyldiisopropylamine), N,N-dimethylbenzylamine; basic inorganic hydroxides, such as, for example, aluminium oxide, calcium oxide, sodium hydroxide, potassium hydroxide, lithium hydroxide; basic ion exchangers, such as, for example, Amberlyst A21.

The concentration of the catalyst, for example of the tertiary amine, is preferably in the range of from 1 to 10 mol %, preferably approximately from 3 to 5 mol %, per mol of compound of formula (III) used. The optimum concentrations for the other catalysts mentioned above can readily be determined by the person skilled in the art.

In order to carry out the present invention, the procedure is preferably as follows: a compound of formula (III) and a catalytic amount of base, for example triethylamine, are dissolved or suspended in an organic solvent. Then acrylic acid alkyl ester is added. The suspension is heated at boiling for approximately from 4 to 6 hours. After cooling to room temperature, the product, that is to say the salt of the compound (I), is filtered off directly, optionally after addition of an antisolvent such as tert-butyl methyl ether. There is thus generally obtained a product having a purity of more than 99% (>99%). The moist product as crude product is preferably dissolved in an organic solvent, while hot, and crystallised by addition of an antisolvent.

There is thus generally obtained a product having a purity of more than 99.5% (>99.5%).

The following examples illustrate the invention without limiting it.

### EXAMPLE 1

Triethylamine (9 mg, 0.09 mmol) and isopropanol (1.5 ml) are added to 0.981 g (3.0 mmol) of the compound of formula (III), wherein R=methyl, R₁=ethyl and HX═HCl. Then methyl acrylate (1.35 ml, 15.0 mmol) is added. The suspension is heated at boiling for 24 hours. After cooling to room temperature (22° C.), the precipitate is filtered off and washed with isopropanol (3 ml). The moist product remifentanil hydrochloride is dried in vacuo (70° C.). 1.03 g (82%) of remifentanil hydrochloride are obtained in the form of a colourless solid, in a purity >99%.

### EXAMPLE 2

Triethylamine (3 mg, 0.03 mmol) and isopropanol (1 ml) are added to 0.327 g (1.0 mmol) of the compound of formula (III), wherein R=methyl, R₁=ethyl and HX═HCl. Then methyl acrylate (0.45 ml, 5.0 mmol) is added. The suspension is heated at boiling for 24 hours. After cooling to room temperature (22° C.), tert-butyl methyl ether (5 ml) is added and stirring is carried out for 1 hour (h) at room temperature (22° C.). Then the precipitate is filtered off and washed with tert-butyl methyl ether (2 ml). The moist product remifentanil crude (339 mg) is taken up in isopropanol (4 ml) and heated at boiling (0.5 h), and clarification by filtration is carried out. The mixture is left to cool overnight (16 h). The precipitate is filtered off and washed with isopropanol (1 ml), and the moist product is dried in vacuo (70° C., 24 h). 267 mg (65%) of dry product remifentanil are obtained in the form of a colourless solid; purity of >99.5%.

### EXAMPLE 3

Potassium carbonate (0.7 mg, 0.005 mmol) and isopropanol (1 ml) are added to 0.327 g (1.0 mmol) of the compound of formula (III), wherein R=methyl, R₁=ethyl and HX═HCl. Then methyl acrylate (0.45 ml, 5.0 mmol) is added. The suspension is heated at boiling for 24 hours (h). After cooling to room temperature (RT), tert-butyl methyl ether (5 ml) is added and stirring is carried out for 1 h at RT. Then the precipitate is filtered off and washed with tert-butyl methyl ether (5 ml). 218 mg (53%) of remifentanil are obtained in the form of a colourless solid; purity >99%.

### EXAMPLE 4

Potassium carbonate (1.4 mg, 0.01 mmol) and methanol (1 ml) are added to 0.327 g (1.0 mmol) of the compound of formula (III), wherein R=methyl, R₁=ethyl and HX═HCl. Then methyl acrylate (0.45 ml, 5.0 mmol) is added. The suspension is heated at boiling for 25 hours (h). After cooling to room temperature (RT), tert-butyl methyl ether (5 ml) is added and stirring is carried out for 2 h at RT. Then the precipitate is filtered off and washed with tort-butyl methyl ether (5 ml). 303 mg (73%) of remifentanil are obtained in the form of a colourless solid; purity >99%.

### EXAMPLE 5

Sodium carbonate (3.2 mg, 0.03 mmol) and isopropanol (3 ml) are added to 0.981 g (3.0 mmol) of the compound of formula (III), wherein R=methyl, R₁=ethyl and HX═HCl. Then methyl acrylate (1.35 ml, 15.0 mmol) is added. The suspension is heated at boiling for 24 hours (h). After cooling to room temperature (RT), tert-butyl methyl ether (15 ml) is added and stirring is carried out for 0.5 h at RT. Then the precipitate is filtered off and washed with tert-butyl methyl ether (6 ml). 1.196 g (97%) of remifentanil are obtained in the form of a colourless solid; purity >99%.

### EXAMPLE 6

Triethylamine (126 mg, 1.25 mmol) and methanol (12.5 ml) are added to 8.17 g (10.0 mmol) of the compound of formula (III), wherein R=methyl, R₁=ethyl and HX═HCl. Then methyl acrylate (11.3 ml, 125 mmol) is added. The solution is heated at boiling for 6 hours (h). After cooling to room temperature (RT), the suspension is stirred for a further 16 h at RT. Then tert-butyl methyl ether (12.5 ml) is added and the suspension is cooled to 0-5° C. After 1 h at that temperature, the precipitate is filtered off and washed with tert-butyl methyl ether (25 ml). The moist product remifentanil crude (9.095 g) is taken up in methanol (25 ml) and heated at boiling (1 h), and clarification by filtration is carried out. After cooling to RT (1 h), tert-butyl methyl ether (25 ml) is added. The suspension is cooled to 0-5° C. and stirred for 1 h at that temperature. The precipitate is filtered off and washed with tert-butyl methyl ether (25 ml), and the moist product is dried in vacuo (50° C., 15 h). 8.522 g (83%) of dry product remifentanil API are obtained in the form of a colourless solid; purity >99.5%.

### EXAMPLE 7

Triethylamine (126 mg, 1.25 mmol) and methanol (12.5 ml) are added to 8.17 g (10.0 mmol) of the compound of formula (III), wherein R=methyl, R₁=ethyl and HX═HCl. Then methyl acrylate (11.3 ml, 125 mmol) is added. The solution is heated for 6 h at an internal temperature of 50° C. After cooling to RT, the suspension is stirred for a further 16 h at RT. Then the suspension is cooled to 0-5° C. After 1 h at that temperature, the precipitate is filtered off and washed with tert-butyl methyl ether (25 ml). The moist product remifentanil crude (8.333 g) is taken up in methanol (25 ml) and heated at boiling (1 h), and clarification by filtration is carried out. After cooling to RT, tert-butyl methyl ether (12.5 ml) is added. The suspension is cooled to 0-5° C. and stirred for 1 h at that temperature. The precipitate is filtered off and washed with tert-butyl methyl ether (25 ml), and the moist product is dried in vacuo (50° C., 18 h). 7.648 g (74%) of dry product remifentanil API are obtained in the form of a colourless solid; purity >99.5%.

### EXAMPLE 8

Methanol (5 ml) and methyl acrylate (4.5 ml, 50 mmol) are added to 3.27 g (10 mmol) of the compound of formula (III), wherein R=methyl, R₁=ethyl and HX═HCl. Heating is carried out for 47.5 h at an internal temperature of 50° C. Then the mixture is cooled to 0-5° C. and the suspension is stirred for 1 h at 0-5° C. Then the precipitate is filtered off and washed with tert-butyl methyl ether (10 ml). 3.381 g (82%) of remifentanil are obtained in the form of a colourless solid; purity >99%.

### EXAMPLE 9

Triethylamine (51 mg, 0.5 mmol) and acetonitrile (5 ml) are added to 3.27 g (10 mmol) of the compound of formula (III), wherein R=methyl, R₁=ethyl and HX═HCl. Then methyl acrylate (4.5 ml, 50 mmol) is added. Heating is carried out for 21.5 h at an internal temperature of 50° C. Then the mixture is cooled to 0-5° C. and the suspension is stirred for 1 h at 0-5° C. Then the precipitate is filtered off and washed with tert-butyl methyl ether (10 ml). 3.617 g (88%) of remifentanil are obtained in the form of a colourless solid; purity >98%.

### EXAMPLE 10

Triethylamine (51 mg, 0.5 mmol) and methanol (5 ml) are added to 3.27 g (10 mmol) of the compound of formula (III), wherein R=methyl, R₁=ethyl and HX═HCl. Then methyl acrylate (4.5 ml, 50 mmol) is added. Stirring is carried out for 18 h at RT. Then the mixture is cooled to 0-5° C. and the suspension is stirred for 45 minutes at 0-5° C. Then the precipitate is filtered off and washed with tert-butyl methyl ether (10 ml). 3.243 g (79%) of remifentanil are obtained in the form of a colourless solid; purity >98%.

### EXAMPLE 11

Triethylamine (51 mg, 0.5 mmol) and methyl acrylate (4.5 ml, 50 mmol) are added to 3.27 g (10 mmol) of the compound of formula (III), wherein R=methyl, R₁=ethyl and HX═HCl. Heating is carried out for 22 h at an internal temperature of 50° C. Then the mixture is cooled to RT and the suspension is stirred for 30 minutes at RT. Then the precipitate is filtered off and washed with tert-butyl methyl ether (10 ml). 3.512 g (85%) of remifentanil are obtained in the form of a colourless solid; purity >97%.

### EXAMPLE 12

Amberlyst A21 (500 mg) and methanol (5 ml) are added to 3.27 g (10 mmol) of the compound of formula (III), wherein R=methyl, R₁=ethyl and HX═HCl. Then methyl acrylate (4.5 ml, 50 mmol) is added. Heating is carried out for 6.5 h at an internal temperature of 50° C. Then hot filtration is carried out at 50° C. The filtrate is cooled to 0-5° C. and the suspension is stirred for 15 minutes at 0-5° C. Then the precipitate is filtered off and washed with tert-butyl methyl ether (10 ml). 2.442 g (59%) of remifentanil are obtained in the form of a colourless solid; purity >99%.

What is claimed is:

1. Process for the preparation of N-phenyl-N-(4-piperidinyl)amide salts, in particular pharmaceutically acceptable addition salts of the compound remifentanil, characterised in that a compound of formula (III) is reacted with an acrylic acid alkyl ester of the formula CH₂═CH—C(O)—OR: 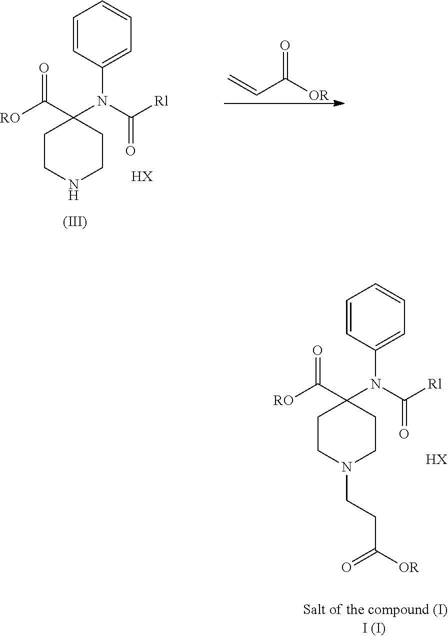
wherein
the substituents R independently of one another denote low-molecular alkyl, preferably (C₁₋₄)-alkyl, preferably methyl or ethyl,
R₁ denotes low-molecular alkyl, preferably (C₁₋₄)-alkyl, preferably methyl or ethyl; and HX denotes an inorganic or organic acid,
wherein the components are optionally reacted in the presence of a catalyst, preferably at elevated temperature, the salt of the compound of formula (I) being obtained.

2. Process according to claim 1, characterised in that R denotes methyl; R₁ denotes ethyl; and HX denotes hydrogen halide, preferably HBr, HI, HCl, preferably HCl; or an organic mono- or di-carboxylic acid, preferably oxalic acid.

3. Process according to claim 1, characterised in that R denotes methyl, R₁ denotes ethyl and HX denotes HCl.

4. Process according to claim 1, characterised in that the compound of formula (III) is reacted with the acrylic acid alkyl ester of the formula CH₂═CH—C(O)—OR and the acrylic acid alkyl ester is used as solvent.

5. Process according to claim 1, characterised in that the compound of formula (III) is reacted with the acrylic acid alkyl ester in a suitable inert solvent, and the solvent is preferably an alcohol, preferably methanol, ethanol, n-propanol, isopropanol, butanol; or an ether, preferably tert-butyl methyl ether; or tetrahydrofuran (THF); or acetonitrile; or a mixture of those compounds.

6. Process according to claim 5, characterised in that the inert solvent or the mixture of inert solvents contains the acrylic acid alkyl ester at least in equimolar amount, preferably in an amount of from 1 to 10 equivalents, preferably in an amount of from 3 to 10 equivalents, preferably in an amount of approximately from 3 to 6 equivalents, and in particular in an amount of approximately 5 equivalents, calculated on the amount of compound of formula (III).

7. Process according to claim 1, characterised in that the reaction is carried out in the presence of a suitable catalyst, and the catalyst is selected from the group comprising: metal carbonates, preferably sodium carbonate, potassium carbonate, lithium carbonate, magnesium carbonate, calcium carbonate; tertiary amines, preferably triethylamine, N-methylmorpholine, Hünig base (ethyldiisopropylamine), N,N-dimethylbenzylamine; basic inorganic hydroxides, preferably aluminium oxide, calcium oxide, sodium hydroxide, potassium hydroxide, lithium hydroxide; basic ion exchangers, preferably Amberlyst A21.

8. Process according to claim 7, characterised in that the concentration of the catalyst is in the range from 1 to 10 mol %, preferably approximately from 3 to 5 mol %, per mol of compound of formula (III) used.

9. Process according to claim 1, characterised in that the reaction mixture is treated at a temperature in the range of approximately from 20 to 120° C., preferably at a temperature in the range of approximately from 50 to 80° C., for approximately from 1 hour to 48 hours, preferably for approximately from 4 to 6 hours.

10. Salts of the compound of formula (I) in crystalline or amorphous form, prepared according to claim 1.

11. Use of the salts of the compound of formula (I) prepared according to claim 1 in the preparation of the free base of the compound of formula (I), by converting the salt of the compound of formula (I) into the free base of the compound of formula (I) in a manner known per se.

12. Process for the preparation of the free base of the compound of formula (I), characterised in that a salt of the compound of formula (I) prepared according to claim 1 is converted into the free base of the compound of formula (I) in a manner known per se.
